(19) United States
(12) Patent Application Publication
(10) Pub. No.: US 2007/0286887 A1
(43) Pub. Date: Dec. 13, 2007

(54) FINE POWDER OF AMINO ACID AND SUSPENSION THEREOF

(75) Inventors: Meguru Kaminoyama, Yokohama-shi (JP); Kazuhiko Nishi, Yokohama-shi (JP); Tohru Kouda, Kawasaki-shi (JP); Susumu Tsujimoto, Kawasaki-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: AJINOMOTO CO., INC, Tokyo (JP)

(21) Appl. No.: 11/759,320

(22) Filed: Jun. 7, 2007

Related U.S. Application Data
(63) Continuation of application No. PCT/JP05/22905, filed on Dec. 7, 2005.

(30) Foreign Application Priority Data
Dec. 7, 2004 (JP) 354391/2004

(51) Int. Cl. A61K 31/198; A61K 47/00

(52) U.S. Cl. 424/439; 514/562

(57) ABSTRACT

Fine powders of a slightly soluble amino acid having an average particle size of 0.1 to 5 μm, may be obtained by wet milling the slightly soluble amino acid. These powders are useful for preparing suspensions in which the fine powder is uniformly and stably dispersed.

### CROSS REFERENCES TO RELATED APPLICATIONS

This application is a continuation of International Patent Application No. PCT/JP2005/022905, filed on Dec. 7, 2005, and claims priority to Japanese Patent Application No. 2004-354391, filed on Dec. 7, 2004, both of which are incorporated herein by reference in their entireties.

### BACKGROUND OF THE INVENTION

### Field of the Invention

The present invention relates to powders of slightly soluble amino acids, such as cystine, aspartic acid, tyrosine, glutamic acid, tryptophan, and the like. The present invention also relates to suspensions comprising such a slightly soluble amino acid, which are uniformly and stably dispersed therein.

### DISCUSSION OF THE BACKGROUND

Amino acids are essential components for the human body and the growth of animals, and new values relating to health functionality in various aspects have been found in recent years in addition to their nutritional value. Accordingly, food, drink, and pharmaceutical products containing amino acid are desirable. For example, cystine ingested over a given amount has been found to be effective for the prevention of infection and the like, and expected to afford health food and health drink. In the case of slightly soluble amino acids having a low solubility in water, however, a necessary amount thereof cannot be dissolved even when addition to a drink is desired, and a uniform and stable dispersion of a slightly soluble amino acid is also difficult to obtain since it generally has high specific gravity (e.g., the absolute specific gravity of cystine is 1.6) and high precipitation property. Accordingly, none of the conventional enteral feeding products and infusions containing poorly water-soluble amino acid contains a large amount of amino acid beyond its solubility.

While attempts have been made by employing methods such as addition of a thickener to increase viscosity of the medium, suppression of precipitation by addition of a fibrous support and the like to a medium, addition of a dispersing agent and the like to achieve uniform dispersion and the like, these methods give rise to problems such as a low mixing property of liquid, low texture for ingestion as a drink, markedly degraded transparency of suspension, easy precipitation upon a treatment such as heating and the like. Alternatively, the stability of the quality may be degraded as evidenced by inconsistent content of a product in the production step and the like. Particularly, when added to a drink, precipitated amino acid causes difficult ingestion, or particularly, a lower quality reliability. This background is discusses in the following references: (1) JP-A-2003-47871; (2) JP-A-2003-47870; (3) Powder performance of prototype continuous annular type wet medium mill (first report), Mitsuo KAMIWANO et al., SHIKIZAI KYOKAISHI, vol. 66, pp. 523-528 (1993); (4) Powder performance of prototype continuous annular type wet medium mill (second report)—influence of various grinding factors on grinding performance—, Mitsuo KAMIWANO et al., SHIKIZAI KYOKAISHI, vol. 66, pp. 529-533 (1993); and (5) Indication and evaluation method of grinding progression state of fine particles in continuous annular type wet medium mill, Mitsuo KAMIWANO et al., Chemical Engineering Papers, vol. 25, pp. 796-802 (1999).

Thus, there remains a need for stable and uniform suspensions which contain a high concentration of a slightly-soluble amino acid.

### SUMMARY OF THE INVENTION

Accordingly, it is one object of the present invention to provide novel suspensions comprising a slightly soluble amino acid.

It is another object of the present invention to provide novel suspensions comprising a high concentration of a slightly soluble amino acid.

It is another object of the present invention to provide novel suspensions comprising a slightly soluble amino acid which are stable.

It is another object of the present invention to provide novel suspensions comprising a slightly soluble amino acid which are uniform.

It is another object of the present invention to provide novel suspensions comprising a high concentration of a slightly soluble amino acid which are stable.

It is another object of the present invention to provide novel suspensions comprising a high concentration of a slightly soluble amino acid which are uniform.

It is another object of the present invention to provide novel powders which are useful for preparing such a suspension.

It is another object of the present invention to provide novel methods for preparing such a powder.

It is another object of the present invention to provide novel methods for preparing such a suspension.

It is another object of the present invention to provide novel products which contain or are prepared from such suspension.

These and other objects, which will become apparent during the following detailed description, have been achieved by the inventors' discovery that a fine powder having an average particle size of not more than a given level can be obtained by wet milling of a crystalline or amorphous slightly soluble amino acid such as cystine, aspartic acid, tyrosine, glutamic acid, tryptophan, and the like, and that such a fine powder shows remarkably lowered tendency to precipitate when suspended, whereby a uniform and stable suspension can be afforded.

Accordingly, the present invention provides the following:

(1) A fine powder of a slightly soluble amino acid having an average particle size of 0.1 to 5 μm.

(2) The fine powder of the above-mentioned (1), wherein the slightly soluble amino acid comprises one or more members selected from the group consisting of cystine, aspartic acid, tyrosine, glutamic acid, tryptophan, and mixtures thereof

(3) The fine powder of the above-mentioned (1), wherein the slightly soluble amino acid comprises cystine.

(4) A suspension, comprising a fine powder of the above-mentioned (1) suspended in a liquid medium.

(5) The suspension of the above-mentioned (4), wherein the slightly soluble amino acid comprises one or more members selected from the group consisting of cystine, aspartic acid, tyrosine, glutamic acid, tryptophan, and mixtures thereof.

(6) The suspension of the above-mentioned (4), wherein the slightly soluble amino acid comprises cystine.

(7) The suspension of any one of the above-mentioned (4) to (6), wherein the concentration of the slightly soluble amino acid is 10 mg/100 g of the suspension to 30 g/100 g of the suspension.

(8) The suspension of any one of the above-mentioned (4) to (7), which further comprises a dispersing agent.

(9) The suspension of the above-mentioned (8), wherein the dispersing agent comprises one or more members selected from the group consisting of lecithin, methylcellulose, pectin, and mixtures thereof.

(10) The suspension of the above-mentioned (8) or (9), wherein the dispersing agent is present in the suspension in an amount of 0.2% to 20% relative to the amount of the suspended amino acid.

(11) A method of producing the suspension of any one of the above-mentioned (4) to 10), which comprises wet milling a slightly soluble amino acid in the presence of water or an organic solvent.

(12) The method of the above-mentioned (11), wherein the milling is performed in the presence of a dispersing agent.

(13) The method of the above-mentioned (12), wherein the dispersing agent is added in an amount of 0.2% to 20% relative to the weight of the suspended amino acid.

(14) A method of producing the fine powder of any one of the above-mentioned (1) to (3), which comprises wet milling a slightly soluble amino acid in the presence of water or an organic solvent, and drying the amino acid by removing the water or organic solvent.

(15) A drink or food comprising the fine powder of any one of the above-mentioned (1) to (3) or the suspension of any one of the above-mentioned (4) to (10).

(16) The drink or food of the above-mentioned (15), wherein the slightly soluble amino acid is cystine.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is a graph showing changes in the cystine particle size distribution depending on the presence or absence of lecithin;

FIG. 2 is a graph showing changes in the cystine particle size distribution of the lecithin-10% system;

FIG. 3 is a graph showing changes (1) in the cystine particle size distribution of the methylcellulose-3% system;

FIG. 4 is a graph showing changes (2) in the cystine particle size distribution of the methylcellulose-3% system; and

FIG. 5 is a graph showing changes in the cystine average particle size depending on the milling treatment time.

### DETAILED DESCRIPTION OF THE PREFERRED EMBODIMENTS

In the present invention, “slightly soluble” refers to an amino acid which is hardly soluble in water, organic solvents such as ethanol and the like, and mixtures thereof. In the present invention, “slightly soluble” means a solubility of not more than about 1 g/100 g solvent (at 20° C.).

Examples of the “slightly soluble amino acid” in the present invention include cystine, aspartic acid, tyrosine, glutamic acid, tryptophan, and the like, and mixtures thereof, preferably cystine, aspartic acid, tyrosine, and mixtures thereof, particularly preferably cystine.

In the present invention, “suspension” means a suspension wherein a fine powder of slightly soluble amino acid of the present invention is dispersed in a medium such as water, ethanol, and the like, or a mixture thereof, preferably an aqueous suspension.

A preferable aqueous suspension in the present invention may contain, besides water as the medium, salts, a buffer, an organic solvent, and the like.

The slightly soluble amino acid before pulverization may be crystalline or amorphous, preferably crystalline.

The “average particle size” of fine powder in the present invention means, for example, a mass median particle diameter d₅₀, which is a minus sieve (%) of 50% as measured using a laser scattering type particle distribution measuring instrument (e.g., SK Microanalyzer PR07100 manufactured by Seishin Enterprises K.K.).

To uniformly and stably disperse a slightly soluble amino acid such as cystine, aspartic acid, tyrosine, glutamic acid, tryptophan, and the like (average particle size about 20 to 50 μm as crystal of amino acid) in an aqueous medium, the present inventors considered forming a fine powder and tried dry-milling. However, the average particle size achieved by dry-milling is about 5 to 10 μm at most, and a poorly water-soluble amino acid having a particle size of this level could not be uniformly and stably dispersed in water. When pulverized beyond this level, unpreferably, amino acid is highly likely to denature (decompose) due to heat generation.

Thus, the present inventors tried wet milling and first found that slightly soluble amino acid such as cystine and the like can be obtained as a fine powder having an average particle size of about 0.1 to 5 μm, and that the fine powder is suitable for forming a stable suspension. The fine powder of the present invention is uniformly dispersed in a medium such as water, ethanol, and the like, or a mixture thereof and the like to give a stable suspension.

In the present invention, as a wet milling machine used for wet milling for the production of a fine powder, any wet milling machine such as an apparatus for rotating a rotating-cylinder or a disk in a cylindrical container, preferably, a continuous annular type beads mill (e.g., spike mill (trade name) manufactured by INOUE MFG., INC. (SHG-4 vessel, volume 860 ml)), in which a cylinder with a protrusion is rotated, can be used.

The pulverizing conditions are, for example, flow (200 ml/min), interior shaft rotation speed (2,900 rpm), beads packing rate 50%, circulation 9 times, temperature: start of pulverization 27° C., average during pulverization 38° C.

The concentration of amino acid in the medium during wet milling is determined from the aspects of industrial efficiency of pulverization and effectiveness during pulverization, and varies depending on the conditions of desired pulverization and the kind of amino acid to be pulverized. The amino acid is added to a medium in a proportion of 0.1 to 30 wt %, preferably about 2 to 20 wt %. When the concentration is higher than this range, the viscosity of the suspension increases and when it is lower, the efficiency of pulverization decreases. The amino acid concentration of a medium can be determined from the weight of amino acid added to the liquid. Alternatively, a sample of the suspension is prepared, which is dissolved by an appropriate method such as addition of acid or alkali, and the like, and the resulting solution is subjected to a general analysis method such as an amino acid analyzer and the like, whereby the amino acid concentration can be determined.

Including dilution after wet milling, the concentration of the slightly soluble amino acid in the suspension is, for example, 10 mg/100 g of suspension to 30 g/100 g of suspension, preferably, 10 mg/100 g of suspension to 20 g/100 g of suspension.

The viscosity can be measured, for example, using a rotary disk viscometer (DVM-B, manufactured by Tokyo Keiki Co., Ltd.), and an apparatus capable of measuring the viscosity of a non-Newtonian liquid by modifying the rotation conditions can also be used. The measurement conditions include the use of, for example, rotor B (30 rpm, 50 sec, 20° C.).

An amino acid crystal generally having an average particle size of about 20 to 50 μm can be processed into a fine powder having an average particle size of about 0.1 to 5 μm, preferably 0.1 to 3.5 μm, by wet milling. In the case of the amino acid, which is a soft organic product permitting an easy increase of viscosity, unlike inorganic products, a further attempt to pulverize finely gives rise to a difficulty such as increased viscosity due to surface friction and the like.

In the present invention, a medium (preferably water) may be added for wet milling. Preferably, a dispersing agent is also added. While the dispersing agent may be added during wet milling, it may be added to a suspension after pulverization rather than during pulverization. When a dispersing agent is added during pulverization, an increase in the viscosity can be suppressed during wet milling, thus enabling finer pulverization, and dispersion stability of a fine powder can be enhanced during preservation.

For example, in Example 2 mentioned below wherein a dispersing agent was not added, the viscosity during pulverization was 94.2 mPa·s (measured for a sample under pulverization), and pulverization was difficult to continue. In contrast, when the dispersing agent (lecithin) shown in Example 3 mentioned below was added, the viscosity during pulverization was 15.7 mPa·s, clearly showing promoted pulverization. The viscosity in these Examples was measured using a rotary disk viscometer (DVM-B, manufactured by Tokyo Keiki CO., Ltd.), and the measurement conditions included the use of rotor B (30 rpm, 50 sec, 20° C.).

Examples of the dispersing agent to be used in the present invention include lecithin, methylcellulose, pectin, and the like, and mixtures thereof, and preferred are lecithin and methylcellulose and mixtures thereof.

The “dispersing agent” refers to a substance whose hydrophobic structure has affinity for a part of the surface of a substance to be suspended, and whose hydrophilic structure has affinity for a solvent for dispersion (e.g., water), thus improving the affinity between the substance to be suspended and the solvent, or a substance that suppresses association of the substances to be suspended, thus improving the uniformity and stability of the suspension. For example, bipolar substances such as lecithin and the like, water-soluble polysaccharides such as methylcellulose and the like, and the like can be mentioned. While the amount of the dispersing agent to be added varies depending on the kind thereof, properties of the solvent, and subject of addition, it is generally about 0.2 to 20%, preferably about 1 to 10%, relative to the weight of the substance to be suspended. Depending on the conditions, addition of a different amount may be appropriate.

Hard particles may be co-present during wet milling. Hard particles have higher hardness than the substance to be pulverized, or comparatively high specific gravity that enables application of a sufficient pulverization energy to substance particles to be substantially pulverized. For example, zirconium can be used. The particle size of hard particles is, for example, 100 to 700 μm, preferably about 400 μm. While a suitable amount to be added varies depending on the conditions of hard particles and the target substance, it is, for example, about a half the volume of the pulverization container.

A suspension after wet milling can be directly or after appropriate dilution added to a drink, other food or a pharmaceutical product and the like.

The fine powder of slightly soluble amino acid of the present invention can be obtained by desolvation or drying of a suspension obtained by wet milling. The drying method varies depending on the kind of the medium of the suspension and the like. Any method, such as drying under reduced pressure, freeze-drying, air drying after casting on a plate and the like, can be used, with preference given to freeze-drying.

The fine powder of the present invention can be added to a drink, other food or a pharmaceutical product and the like. In addition, various preparations can be produced by a general preparation technique comprising appropriate granulation processing to give granules and the like, or filling in a capsule and the like.

The amount of the fine powder and/or suspension of the present invention to be added to a drink, other food or a pharmaceutical product and the like is an amount necessary and sufficient for each amino acid to express its given function. Especially when added to a drink, a liquid food or a liquid pharmaceutical product, each amino acid is stably suspended in a fine particle state. Therefore, transparency and low viscosity can be maintained even at a high concentration, which is preferable from the aspect of preference.

The suspension and fine powder of the present invention contain an amino acid in a fine particle state, and can stably and remarkably improve bowel absorption in mammals, including humans. Therefore, they can effectively and stably maintain a particular function or treatment effect, and are useful for the maintenance of good health or therapy.

Other features of the invention will become apparent in the course of the following descriptions of exemplary embodiments which are given for illustration of the invention and are not intended to be limiting thereof.

### EXAMPLES

The spike mill used in the Examples was SHG-4 manufactured by INOUE MFG., INC. (pulverization air volume 75 ml, vessel volume 860 ml), and the treatment conditions were 200 ml/minute, interior shaft rotation speed 2,900 rpm, beads diameter 400 μm. The particle size distribution was measured using a laser scattering type particle distribution measuring instrument (manufactured by Seishin Enterprises K.K.: SK Microanalyzer PRO7100). For the dispersion stability of a suspension, the suspension and a 10-fold diluted solution thereof were stood still, and the precipitation state (precipitation rate, precipitate volume) of amino acid was observed.

### Example 1

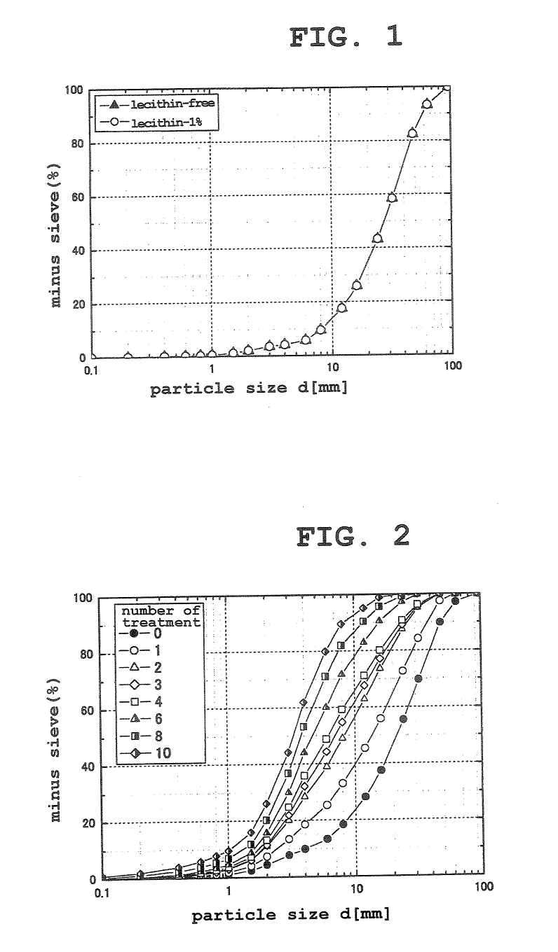

The difference in particle size distribution was examined based on the presence or absence of a dispersing agent. A spike mill was not used. A sample contained cystine (600 g), lecithin (0 g or 6 g), and water (5400 g). FIG. 1 shows the particle size distribution (average particle size 25 μm) when lecithin was not added and when 1 wt % of lecithin was added to cystine. They showed similar results where a mere addition of a dispersing agent did not change the particle size. Therefore, it was clarified that both were not aggregates in which cystine could be broken by the dispersing agent, but instead require division into fine particles by pulverization for stable suspending.

### Example 2

A sample containing cystine (600 g) relative to water (5400 g) was prepared and treated with a spike mill.

Although the viscosity increased as the number of treatments increased, the average particle size d₅₀ became smaller, which can be evaluated to show division into fine particles in progress. Particles having an average particle size d₅₀ of 3 μm could be obtained by 9 repetitions of treatment. The suspension after pulverization was stable for about 1 week and did not show any precipitation.

### Example 3

A sample containing cystine (600 g) and lecithin (60 g) (10 wt % relative to cystine) and water (5400 g) was prepared and treated with a spike mill in the same manner as in Example 2.

FIG. 2 shows changes in the particle size distribution when the sample was subjected to a dispersion test. The average particle size d₅₀ became smaller as the number of treatments increased, which can be evaluated to show division into fine particles in progress. Since an increase in the viscosity was small as the number of treatments increased, the addition of lecithin is considered to have promoted the pulverization.

The dispersion stability of a diluted solution obtained by adjusting the suspension (number of treatments 9) to 1% concentration showed improvement since the volume of precipitate decreased as compared to non-addition of lecithin. However, the time necessary for the precipitation was found to be short. This is considered to be attributable to the suppressed increase in the viscosity of the dispersion.

However, when this was added to a commercially available drink (Calpis Water (registered trademark)) containing a dispersing agent such as pectin and the like (final 0.3% relative to solution), precipitation was not observed after standing still for not less than 1 day. It was shown that a smaller particle size could afford practical dispersion stability.

### Example 4

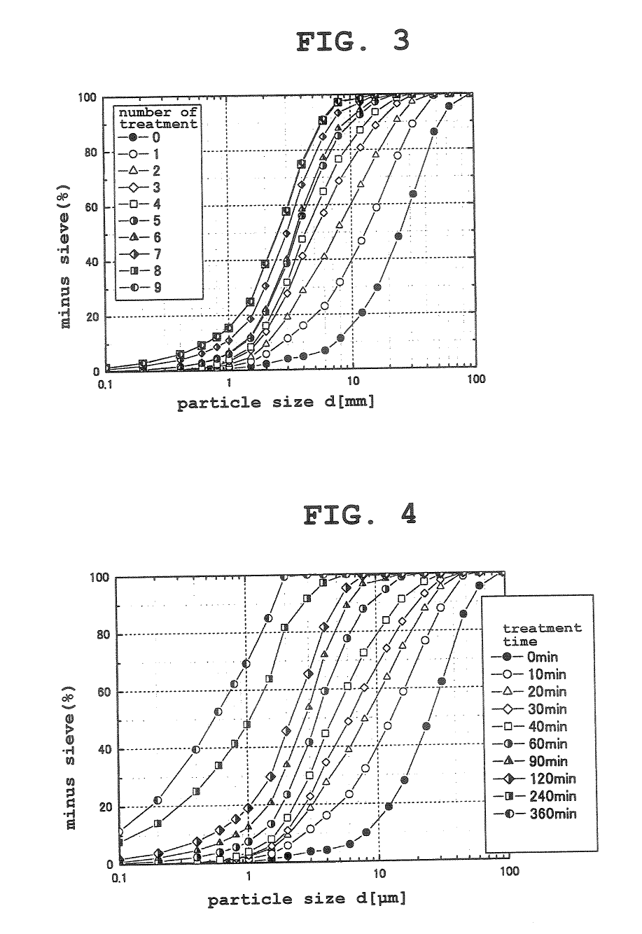

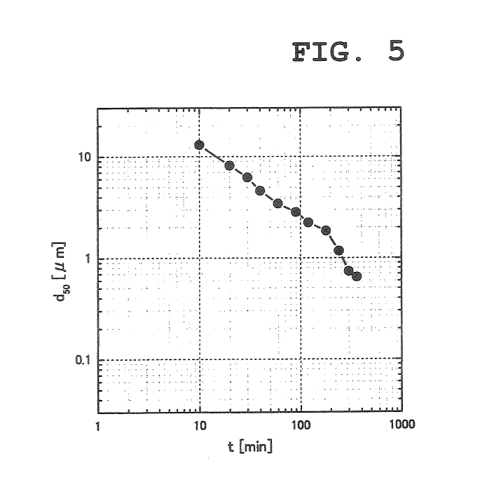

A sample containing cystine (600 g) and methylcellulose (18 g, 3% relative to cystine) and water (5400 g) was prepared and treated with a spike mill in the same manner as in Example 2. With regard to a dispersion experiment using methylcellulose as a dispersing agent, FIG. 3 shows changes in the particle size distribution for each number of treatments, FIG. 4 shows changes in the particle size distribution for each treatment time, and FIG. 5 shows an average particle size profile for the treatment time.

Particles having an average particle size d₅₀ of 2.4 μm could be obtained by 8 repetitions of treatment. The viscosity hardly increased as the number of treatments increased. The suspension after pulverization was stable for about 1 week and did not show any precipitation.

As regards the dispersion stability of a diluted solution obtained by adjusting the suspension to 1% concentration, the amount of precipitation was lowered as compared to non-addition of a dispersing agent or use of lecithin instead of methylcellulose as a dispersing agent. The solution was mostly stable under the conditions of standing still for not less than 1 day. The stability was further improved by the addition of a dispersing agent such as pectin and the like.

### INDUSTRIAL APPLICABILITY

According to the present invention, useful slightly soluble amino acids such as cystine, aspartic acid, tyrosine, glutamic acid, tryptophan, and the like, which have been conventionally difficult for addition at a high concentration from the aspects of solubility and suspending property, can be uniformly and stably suspended in a drink, a liquid food or a pharmaceutical product and the like, and a suspension of the amino acid can be effectively used for a drink, a liquid food, a pharmaceutical product and the like. The suspension of the present invention can be used for the production of a drink, a liquid food, a pharmaceutical product and the like, which contain nutrition and a slightly soluble amino acid in an amount necessary and sufficient for the expression of nutrition and a particular function, at a high concentration and in a uniform dispersion state. Moreover, since the slightly soluble amino acid fine powder or suspension of the present invention is superior in bowel absorption in mammals, including humans, and can effectively express a desired nutrition and a particular function, it is extremely useful in the fields of food and pharmaceutical products.

Using the suspension of the present invention, for example, an amino acid superior in nutrition and particular health function can be contained in a drink or food, especially a drink having a low viscosity and high transparency such as tea, sports drink, and the like, or a food required to have transparency as well as texture such as jelly, gelidium jelly and the like, in a sufficient amount necessary from the aspects of nutrition and health. In addition, realization of uniform liquid dispersion state in a low viscosity liquid is also important for the quality management aiming to contain a given amount of amino acid indicated on each package unit in the production process of a food, drink or pharmaceutical product.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A powder, comprising a slightly soluble amino acid, wherein said slightly soluble amino acid has an average particle size of 0.1 to 5 μm.

2. The powder of claim 1, wherein said slightly soluble amino acid comprises one or more members selected from the group consisting of cystine, aspartic acid, tyrosine, glutamic acid, tryptophan, and mixtures thereof.

3. The powder of claim 1, wherein said slightly soluble amino acid comprises cystine.

4. A suspension, comprising a powder of claim 1 suspended in a liquid medium.

5. The suspension of claim 4, wherein said slightly soluble amino acid comprises one or more members selected from the group consisting of cystine, aspartic acid, tyrosine, glutamic acid, tryptophan, and mixtures thereof.

6. The suspension of claim 4, wherein said slightly soluble amino acid comprises cystine.

7. The suspension of claim 4, wherein said suspension comprises said slightly soluble amino acid in a concentration of 10 mg/100 g of suspension to 30 g/100 g of suspension.

8. The suspension of claim 5, wherein said suspension comprises said slightly soluble amino acid in a concentration of 10 mg/100 g of suspension to 30 g/100 g of suspension.

9. The suspension of claim 6, wherein said suspension comprises said slightly soluble amino acid in a concentration of 10 mg/100 g of suspension to 30 g/100 g of suspension.

10. The suspension of claim 4, which further comprises a dispersing agent.

11. The suspension of claim 5, which further comprises a dispersing agent.

12. The suspension of claim 6, which further comprises a dispersing agent.

13. The suspension of claim 7, which further comprises a dispersing agent.

14. The suspension of claim 10, wherein said dispersing agent comprises one or more members selected from the group consisting of lecithin, methylcellulose, pectin, and mixtures thereof.

15. The suspension of claim 10, wherein said dispersing agent is present in said suspension in an amount of 0.2% to 20% relative to the weight of the suspended amino acid.

16. A method of producing the suspension of claim 4, which comprises wet milling a slightly soluble amino acid in the presence of water or an organic solvent.

17. The method of claim 16, wherein said milling is performed in the presence of a dispersing agent.

18. The method of claim 17, wherein said dispersing agent is present in an amount of 0.2% to 20% relative to the weight of the suspended amino acid.

19. A method of producing a fine powder of claim 1, which comprises:
(i) wet milling a slightly soluble amino acid in the presence of water or an organic solvent, to obtain a milled amino acid; and
(ii) drying said milled amino acid by removing the water or organic solvent.

20. A drink or food, comprising a fine powder of claim 1.

21. The drink or food of claim 15, wherein said slightly soluble amino acid comprises cystine.
